(19) United States
(12) Patent Application Publication
(10) Pub. No.: US 2024/0261256 A1
(43) Pub. Date: Aug. 8, 2024

(54) METHODS OF TREATING BACTERIAL INFECTIONS

(71) Applicants: Sheba Impact Ltd., Ramat-Gan (IL); Ministry of Health, State of Israel, Jerusalem (IL)

(72) Inventors: Yaacov DAVIDOV, Jerusalem (IL); Galia RAHAV, Jerusalem (IL); Alla USYSKIN-TONNE, Rishon Le-Zion (IL); Alex SHULMAN, Rishon LeZion (IL); Ari ROBINSON, Bnei-Atarot (IL); Israel Ronen NISSAN, ModiIn (IL); Noa Lea TEJMAN-YARDEN, Tel-Aviv (IL)

(73) Assignees: Sheba Impact Ltd., Ramat-Gan (IL); Ministry of Health, State of Israel, Jerusalem (IL)

(21) Appl. No.: 18/101,611

(22) Filed: Jan. 26, 2023

(51) Int. Cl. A61K 31/357; A61K 31/4025; A61K 31/4433; A61K 33/24; A61P 31/04

(52) U.S. Cl. CPC A61K 31/357; A61K 31/4025; A61K 31/4433; A61K 33/24; A61P 31/04

(57) ABSTRACT

A method of treating an infection of a gram positive bacteria in a subject in need thereof is disclosed. The method comprises administering to the subject a therapeutically effective amount of enterobactin and/or an active derivative thereof. Compositions comprising enterobactin are also disclosed or active derivatives are also disclosed.

### FIELD AND BACKGROUND OF THE INVENTION

The present invention, in some embodiments thereof, relates to the antimicrobial effect of enterobactin and derivatives thereof against bacteria and more particularly, against gram positive bacteria.

The excessive global use of antimicrobial drugs in humans as well as agriculture and veterinary medicine has led to the emergence of new multidrug resistant pathogens. Many of these pathogens are globally spread and treatment options for clinicians are becoming limited.

Among the Gram positive “spectrum”, bacteria posing threats of resistance are the methicillin resistant Staphylococcus aureus (MRSAs). The MRSAs are traditionally divided into community acquired MRSA (CA-MRSA) and hospital acquired MRSA (HA-MRSA). The HA-MRSA are in general more resistant than CA-MRSA due to a larger “Staphylococcal Chromosomal Cassette” region (SCCmec). The CA-MRSA clone USA300 evolved in the USA in year 2000. Within a few years it has spread extensively across the USA, effectively marginalizing other S. aureus strains, MRSA as well as MSSA. This successful and virulent clone has gained a worldwide distribution. It is mainly community-associated, but hospital-associated cases also occur. Another example is the vancomycin-resistant enterococci which are prevalent across the globe. Due to the increase in factors predisposing the risk of infection and carriage of resistance to today's' arsenal of antibiotics, there is an increasing demand for new compounds with antibacterial activity which bypass/overcome resistance.

Siderophores are low molecular weight, high-affinity iron chelators that are biosynthesized and exported by bacteria, fungi and plants during periods of nutrient limitation for acquiring this essential metal ion from the extracellular milieu. Sideromycins are a unique type of antibiotics which are covalently linked to siderophores, such as albomycin and salmycin.

Enterobactin and salmochelin are siderophores produced by Gram-negative bacteria such as E. coli, Salmonella enterica and Klebsiella pneumoniae. They enable scavenging of Fe³⁺ from the host environment, are recognized and transported into the cell by an uptake machinery. Salmochelins are di-glucosylated analogs of enterobactin, which are able to evade the host defense protein—lipocalin-2, an enterobactin scavenger (10). For example, the uropathogenic E. coli (UPEC) CFT073 is able to produce enterobactin and salmochelin. Enterobactin—iron complex is transported into CFT073 by the FepA transporter and salmochelin—iron complex by the IroN transporter.

Based on the natural sideromycin, several research groups have synthetized siderophores fused to antibiotics. This “Trojan Horse delivery strategy” proved to be an efficient means for active concentration of antibiotics inside target bacteria (Wilson, B. R., et al., 2016, Trends Mol Med 22, 1077-1090). The antibiotic cefiderocol (Fetroja) is an example of such a drug, which was approved by the U.S. Food and Drug Administration (FDA) and is in clinical use today mostly against Escherichia coli, Klebsiella pneumoniae, Proteus mirabilis, Pseudomonas aeruginosa, Enterobacter cloacae complex and Acinetobacter baumannii (AlMatar, M., et al., (2020) Mini Rev Med Chem 20, 1908-1916).

Additional background art includes US Application No. 20160022794 and International Patent Application No. WO 2015/057958.

### SUMMARY OF THE INVENTION

According to an aspect of the present invention there is provided a method of treating an infection of a gram positive bacteria in a subject in need thereof, the method comprising administering to the subject a therapeutically effective amount of enterobactin and/or an active derivative thereof, thereby treating the infection.

According to some embodiments of the invention, the method comprises administering to the subject a therapeutically effective amount of enterobactin and an active derivative thereof.

According to some embodiments of the invention, the active derivative is a glucosylated derivative.

According to some embodiments of the invention, the gram positive bacteria is of the genus Staphylococcus or Enterococcus.

According to some embodiments of the invention, the method further comprises administering to the subject an antimicrobial agent distinct of the enterobactin and/or the active derivative thereof.

According to some embodiments of the invention, the antimicrobial agent is selected from the group consisting of an antibiotic, a metal and delftibactin.

According to some embodiments of the invention, the enterobactin and/or the active derivative of enterobactin is not conjugated to an antibiotic.

According to some embodiments of the invention, the antibiotic is lincomycin.

According to some embodiments of the invention, the administering is effected concurrently.

According to some embodiments of the invention, the enterobactin and/or the derivative is not conjugated to the antimicrobial agent.

According to some embodiments of the invention, the antimicrobial agent is a metal.

According to some embodiments of the invention, the metal is selected from the group consisting of copper, iron, zinc, gold and gallium.

According to some embodiments of the invention, the glucosylated derivative comprises at least one sugar moiety attached to the C5 position of one or more catecholate rings of the enterobactin.

According to some embodiments of the invention, the glucosylated derivative is Salmochelin S4.

According to an aspect of the present invention there is provided a composition comprising:
(i) enterobactin and/or a glucosylated derivative thereof; and
(ii) delftibactin.

According to some embodiments of the invention, the composition further comprises a pharmaceutically acceptable carrier.

According to some embodiments of the invention, the composition further comprises an antibiotic and/or a metal.

According to some embodiments of the invention, the antibiotic is lincomycin.

According to some embodiments of the invention, the metal is selected from the group consisting of copper, iron, zinc, gold and gallium.

According to an aspect of the present invention there is provided an article of manufacture comprising:
(i) enterobactin and/or a glucosylated derivative thereof; and
(ii) delftibactin.

According to some embodiments of the invention, the article of manufacture further comprises an antibiotic and/or a metal.

According to some embodiments of the invention, the antibiotic is lincomycin.

According to some embodiments of the invention, the metal is selected from the group consisting of copper, iron, zinc, gold and gallium.

According to an aspect of the present invention there is provided a method of killing a bacteria, the method comprising contacting the bacteria with a composition comprising:
(i) enterobactin and an active derivative thereof,
(ii) enterobactin or an active derivative thereof and a metal; or
(iii) enterobactin or an active derivative thereof and an antibiotic.

wherein neither the enterobactin nor the active derivative is conjugated to the antibiotic, thereby killing the bacteria.

According to some embodiments of the invention, the contacting is effected in vivo.

According to some embodiments of the invention, the contacting is effected ex vivo.

According to some embodiments of the invention, the bacteria is a gram positive bacteria.

According to some embodiments of the invention, the bacteria is a gram negative bacteria.

According to some embodiments of the invention, the active derivative is a glucosylated derivative.

According to some embodiments of the invention, the glucosylated derivative comprises at least one sugar moiety attached to the C5 position of one or more catecholate rings of the enterobactin.

According to some embodiments of the invention, the glucosylated derivative is Salmochelin S4.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B are graphs illustrating that Salmochelin (FIG. 1A) and Enterobactin (FIG. 1B) have an inhibitory effect on clinical isolates of MRSA USA300.

FIGS. 2A-B are graphs illustrating that Enterobactin has an inhibitory effect on clinical isolates of VRE 195 (FIG. 2A) and VRE 197 (FIG. 2B).

FIGS. 3A-B are graphs illustrating that Salmochelin has an inhibitory effect on clinical isolates of VRE 195 (FIG. 3A) and VRE 197 (FIG. 3B).

FIG. 4 is a graph illustrating that the combination of Salmochelin and Enterobactin display enhanced activity against S. aureus ATCC25923.

FIG. 5 is a graph illustrating that the combination of Enterobactin and Delftibactin A displays enhanced activity against S. aureus.

FIGS. 6A-B are graphs illustrating that iron ions reduce, but do not abolish, antimicrobial activity of Salmochelin and Enterobactin against S. aureus.

FIG. 7 is a graph illustrating that the combination of Salmochelin or Enterobactin with lincomycin displays enhanced activity against S. aureus.

FIG. 8 is a graph illustrating the effect of gold plus Enterobactin or Salmochelin on E. coli CFT073.

FIGS. 9A-C are graphs illustrating the effect of Enterobactin and gold (6 hours incubation) on Klebsiella pneumonia (KPC) (A), Pseudomonas aeruginosa (B) and Acinetobacter baumannii (C).

FIGS. 10A-C illustrate the structure of enterobactin, salmochelin S4 and Delftibactin A.

### DESCRIPTION OF SPECIFIC EMBODIMENTS OF THE INVENTION

The present invention, in some embodiments thereof, relates to the antimicrobial effect of enterobactin and derivatives thereof against bacteria and more particularly, against gram positive bacteria.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

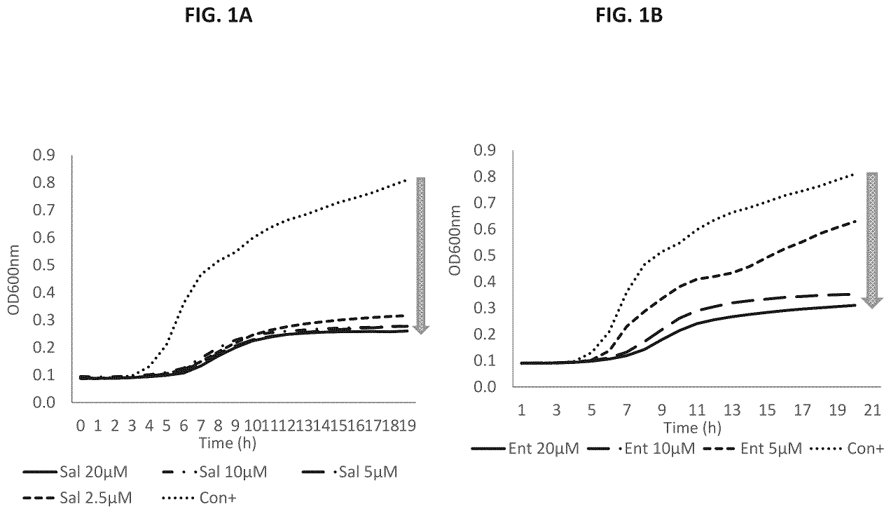

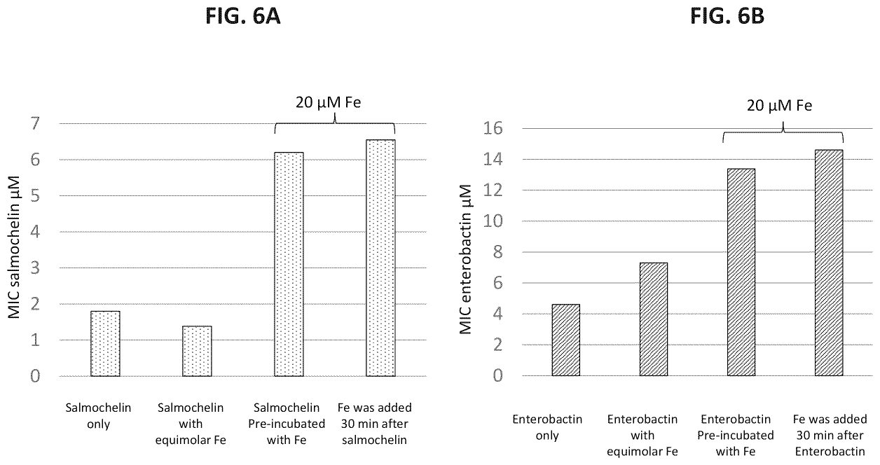

Whilst studying the antimicrobial effect of enterobactin and salmochelin, the present inventors found that these siderophores show anti-microbial activity towards gram positive bacteria, including Staphylococcus aureus (MRSA, MSSA) and vancomycin resistant Enterococci (VRE) (FIGS. 1A-B, 2A-B and 3A-B). This activity remained even in the presence of iron (FIGS. 6A-B).

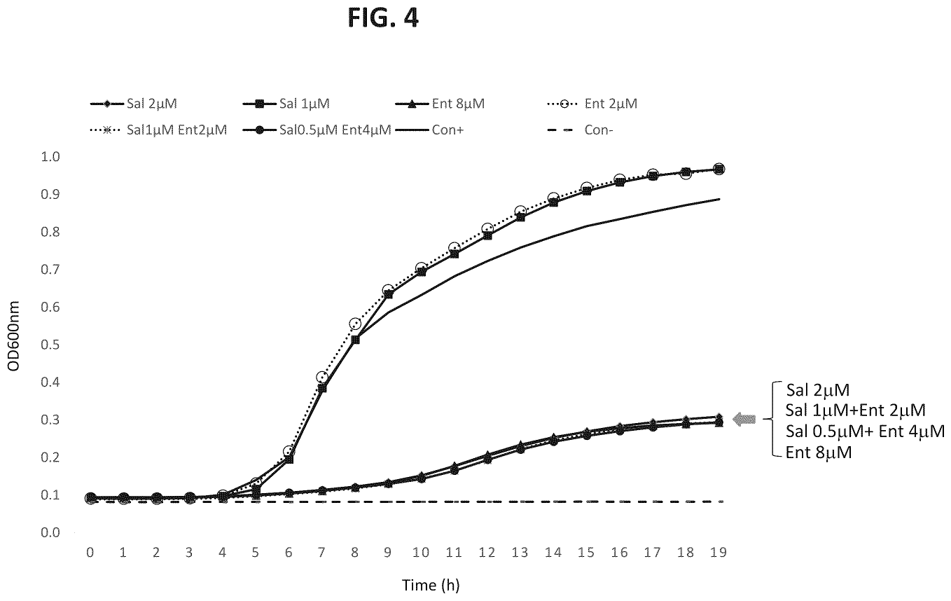

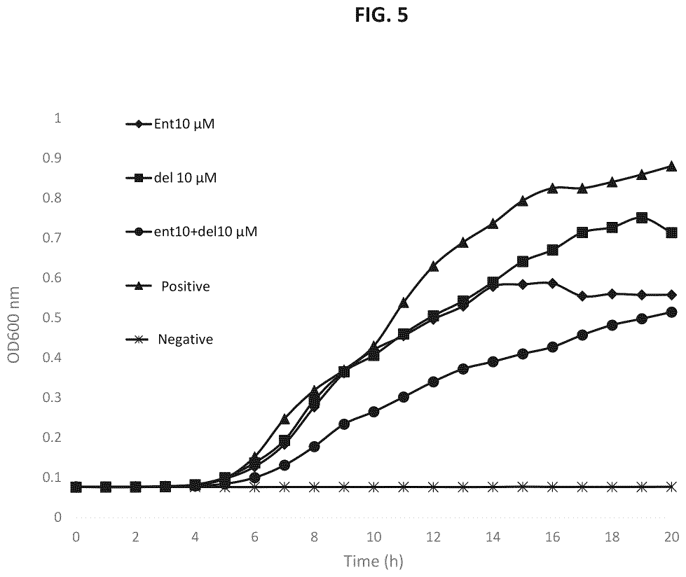

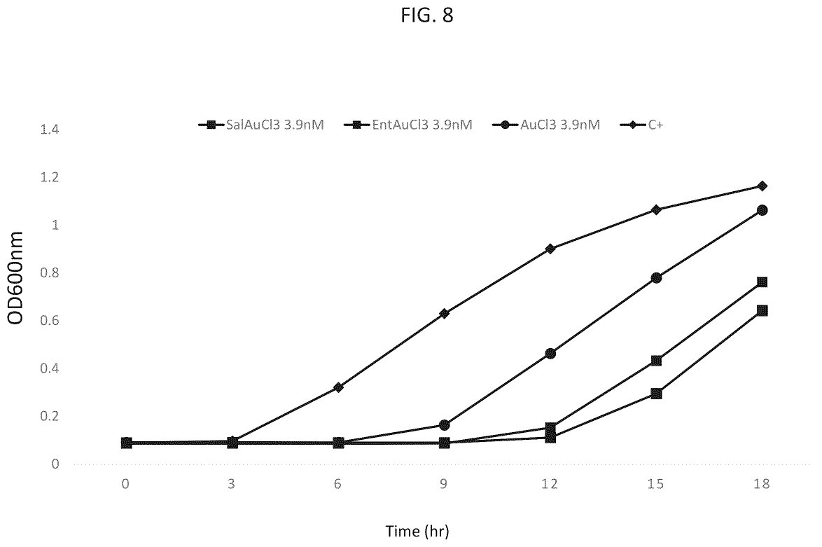

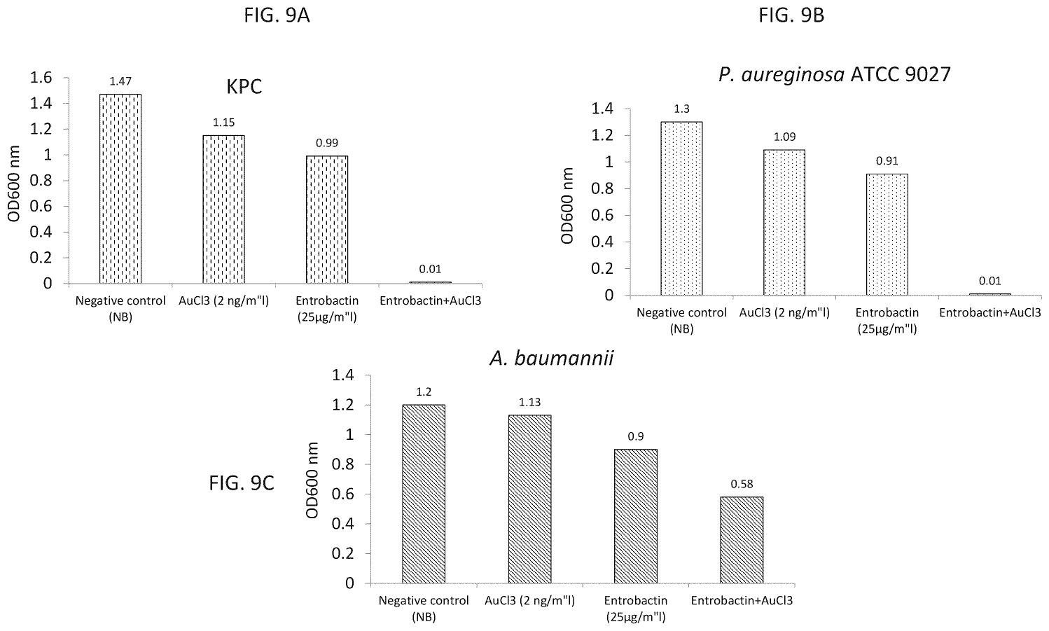

Whilst further reducing the present invention to practice, the present inventors showed that the combination of enterobactin together with salmochelin significantly improved their inhibitory activity (FIG. 4). Similarly, combination of enterobactin with the siderophore delftibactin A, has a synergistic antimicrobial effect (FIG. 5). Combinations of salmochelin and enterobactin with toxic metal ions such as gold and silver, exhibited potent activity against Gram-negative bacteria (FIG. 8, FIGS. 9A-C).

The present inventors propose that enterobactins and other active derivatives thereof (individually or in combination) can serve as a new platform in the battle against Gram positive and Gram negative pathogens.

Thus, according to one aspect of the present invention there is provided a method of treating an infection of a gram positive bacteria in a subject in need thereof, the method comprising administering to the subject a therapeutically effective amount of enterobactin and/or an active derivative thereof, thereby treating the infection.

As used herein, the term “treating” refers to curing, reversing, attenuating, alleviating, minimizing, suppressing or halting the deleterious effects of a pathogen infection.

The terms “administer,” “administering,” or “administration” refers to injecting, implanting, absorbing, ingesting, or inhaling an agent described herein, or a pharmaceutical composition thereof.

Subjects which may be treated according to this aspect of the present invention include mammalian subjects—e.g. humans.

According to one embodiment, the infection is an acute infection.

According to another embodiment, the infection is a chronic infection.

The term “enterobactin” collectively describes compounds having a general formula I:

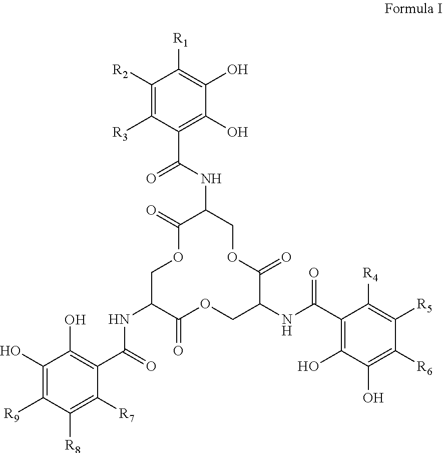

wherein:
R₁-R₉ are each independently selected from hydrogen, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, amino, alkylene glycol, poly(alkylene glycol), and a saccharide moiety, as these groups and moieties are defined herein, or alternatively, two of R₁-R₃, two of R₄-R₆ and/or two of R₇-R₉ together form a five- or six-membered aromatic (aryl or heteroaryl) or alicyclic (cycloalkyl or heteroalicyclic) ring, fused to the respective catecholate ring,
and includes pharmaceutically acceptable salts, hydrates, solvates, polymorphs, co-crystals, tautomers, stereoisomers, and isotopically labeled derivatives thereof, as defined herein.

As used herein throughout, the term “alkyl” describes any saturated aliphatic hydrocarbon including straight chain and branched chain groups. Preferably, the alkyl group has 1 to 20 carbon atoms. Whenever a numerical range; e.g., “1 to 20”, is stated herein, it implies that the group, in this case the hydrocarbon, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 4 carbon atoms. The alkyl group may be substituted or non-substituted. When substituted, the substituent group can be, for example, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

Herein, the term “alkenyl” describes an unsaturated aliphatic hydrocarbon comprise at least one carbon-carbon double bond, including straight chain and branched chain groups. Preferably, the alkenyl group has 2 to 20 carbon atoms. More preferably, the alkenyl is a medium size alkenyl having 2 to 10 carbon atoms. Most preferably, unless otherwise indicated, the alkenyl is a lower alkenyl having 2 to 4 carbon atoms. The alkenyl group may be substituted or non-substituted. Substituted alkenyl may have one or more substituents, whereby each substituent group can independently be, for example, alkynyl, cycloalkyl, alkynyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino.

Herein, the term “alkynyl” describes an unsaturated aliphatic hydrocarbon comprise at least one carbon-carbon triple bond, including straight chain and branched chain groups. Preferably, the alkynyl group has 2 to 20 carbon atoms. More preferably, the alkynyl is a medium size alkynyl having 2 to 10 carbon atoms. Most preferably, unless otherwise indicated, the alkynyl is a lower alkynyl having 2 to 4 carbon atoms. The alkynyl group may be substituted or non-substituted. Substituted alkynyl may have one or more substituents, whereby each substituent group can independently be, for example, cycloalkyl, alkenyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino.

A “cycloalkyl” group describes a saturated on unsaturated all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein. When a cycloalkyl group is unsaturated, it may comprise at least one carbon-carbon double bond and/or at least one carbon-carbon triple bond.

An “aryl” group describes an all-carbon monocyclic or fused-ring polycyclic (i.e., rings which share adjacent pairs of carbon atoms) having a completely conjugated pi-electron system. Examples, without limitation, of aryl groups are phenyl, naphthalenyl and anthracenyl. The aryl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

A “heteroaryl” group describes a monocyclic or fused ring (i.e., rings which share an adjacent pair of atoms) having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

A “heteroalicyclic” group describes a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or non-substituted. When substituted, the substituted group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein. Representative examples are piperidine, piperazine, tetrahydrofuran, tetrahydropyran, morpholine and the like.

Herein, the terms “amine” and “amino” each refer to either a —NR′R″ group or a —N⁺R′R″R′″ group, wherein R′, R″ and R′″ are each hydrogen or a substituted or non-substituted alkyl, alkenyl, alkynyl, cycloalkyl, heteroalicyclic (linked to amine nitrogen via a ring carbon thereof), aryl, or heteroaryl (linked to amine nitrogen via a ring carbon thereof), as defined herein. Optionally, R′, R″ and R′″ are hydrogen or alkyl comprising 1 to 4 carbon atoms. Optionally, R′ and R″ (and R′″, if present) are hydrogen. When substituted, the carbon atom of an R′, R″ or R′″ hydrocarbon moiety which is bound to the nitrogen atom of the amine is not substituted by oxo (unless explicitly indicated otherwise), such that R′, R″ and R′″ are not (for example) carbonyl, C-carboxy or amide, as these groups are defined herein.

An “azide” group describes a —N═N⁺═N group.

An “alkoxy” group describes any of an —O-alkyl, —O-alkenyl, —O-alkynyl, —O-cycloalkyl, and —O-heteroalicyclic group, as defined herein.

An “aryloxy” group describes both an —O-aryl and an —O-heteroaryl group, as defined herein.

A “hydroxy” group describes a —OH group.

A “thiohydroxy” or “thiol” group describes a —SH group.

A “thioalkoxy” group describes any of an —S-alkyl, —S-alkenyl, —S-alkynyl, —S-cycloalkyl, and —S-heteroalicyclic group, as defined herein.

A “thioaryloxy” group describes both an —S-aryl and an —S-heteroaryl group, as defined herein.

A “carbonyl” or “acyl” group describes a —C(═O)—R′ group, where R′ is defined as hereinabove.

A “thiocarbonyl” group describes a —C(═S)—R′ group, where R′ is as defined herein.

A “C-carboxy” group describes a —C(═O)—O—R′ group, where R′ is as defined herein.

An “O-carboxy” group describes an R′C(═O)—O— group, where R′ is as defined herein.

A “carboxylic acid” group describes a —C(═O)OH group.

An “oxo” group describes a ═O group.

An “imine” group describes a ═N—R′ group, where R′ is as defined herein.

An “oxime” group describes a ═N—OH group.

A “hydrazone” group describes a ═N—NR′R″ group, where each of R′ and R″ is as defined herein.

A “halo” group describes fluorine, chlorine, bromine or iodine.

A “sulfinyl” group describes an —S(═O)—R′ group, where R′ is as defined herein.

A “sulfonyl” group describes an —S(═O)₂—R′ group, where R′ is as defined herein.

A “sulfonate” group describes an —S(═O)₂—O—R′ group, where R′ is as defined herein.

A “sulfate” group describes an —O—S(═O)₂—O—R′ group, where R′ is as defined as herein.

A “sulfonamide” or “sulfonamido” group encompasses both S-sulfonamido and N-sulfonamido groups, as defined herein.

An “S-sulfonamido” group describes a —S(═O)₂—NR′R″ group, with each of R′ and R″ as defined herein.

An “N-sulfonamido” group describes an R'S(═O)₂—NR″— group, where each of R′ and R″ is as defined herein.

An “O-carbamyl” group describes an —OC(═O)—NR′R″ group, where each of R′ and R″ is as defined herein.

An “N-carbamyl” group describes an R′OC(═O)—NR″— group, where each of R′ and R″ is as defined herein.

An “O-thiocarbamyl” group describes an —OC(═S)—NR′R″ group, where each of R′ and R″ is as defined herein.

An “N-thiocarbamyl” group describes an R′OC(═S)NR″— group, where each of R′ and R″ is as defined herein.

An “S-thiocarbamyl” group describes an —SC(═O)—NR′R″ group, where each of R′ and R″ is as defined herein.

An “amide” or “amido” group encompasses C-amido and N-amido groups, as defined herein.

A “C-amido” group describes a —C(═O)—NR′R″ group, where each of R′ and R″ is as defined herein.

An “N-amido” group describes an R′C(═O)—NR″— group, where each of R′ and R″ is as defined herein.

A “urea group” describes an —N(R′)—C(═O)—NR″R′″ group, where each of R′, R″ and R″ is as defined herein.

A “thiourea group” describes a —N(R′)—C(═S)—NR″R′″ group, where each of R′, R″ and

R″ is as defined herein.

A “nitro” group describes an —NO₂ group.

A “cyano” group describes a —C≡N group.

The term “phosphonyl” or “phosphonate” describes a —P(═O)(OR′)(OR″) group, with R′ and R″ as defined hereinabove.

The term “phosphate” describes an —O—P(═O)(OR′)(OR″) group, with each of R′ and R″ as defined hereinabove.

The term “phosphinyl” describes a —PR′R″ group, with each of R′ and R″ as defined hereinabove.

The term “hydrazine” describes a —NR′—NR″R′″ group, with R′, R″, and R′″ as defined herein.

As used herein, the term “hydrazide” describes a —C(═O)—NR′—NR″R′″ group, where R′, R″ and R′″ are as defined herein.

As used herein, the term “thiohydrazide” describes a —C(═S)—NR′—NR″R′″ group, where R′, R″ and R′″ are as defined herein.

A “guanidinyl” group describes an —RaNC(═NRd)-NRbRc group, where each of Ra, Rb, Rc and Rd can be as defined herein for R′ and R″.

A “guanyl” or “guanine” group describes an RaRbNC(═NRd)- group, where Ra, Rb and Rd are as defined herein.

As used herein, the phrase “pharmaceutically acceptable salt” describes a charged species of the parent compound and its counter-ion, which is typically used to modify the solubility characteristics of the parent compound and/or to reduce any significant irritation to an organism by the parent compound, while not abrogating the biological activity and properties of the administered compound. A pharmaceutically acceptable salt of a compound as described herein can alternatively be formed during the synthesis of the compound, e.g., in the course of isolating the compound from a reaction mixture or re-crystallizing the compound.

In the context of some of the present embodiments, a pharmaceutically acceptable salt of the compounds described herein may optionally be an acid addition salt and/or a base addition salt.

An acid addition salt comprises at least one basic (e.g., amine and/or amide) group of the compound which is in a positively charged form (e.g., wherein the basic group is protonated), in combination with at least one counter-ion, derived from the selected acid, that forms a pharmaceutically acceptable salt. The acid addition salts of the compounds described herein may therefore be complexes formed between one or more basic groups of the compound and one or more equivalents of an acid.

A base addition salt comprises at least one acidic (e.g., hydroxy, carboxylic acid) group of the compound which is in a negatively charged form (e.g., wherein the acidic group is deprotonated), in combination with at least one counter-ion, derived from the selected base, that forms a pharmaceutically acceptable salt. The base addition salts of the compounds described herein may therefore be complexes formed between one or more acidic groups of the compound and one or more equivalents of a base.

Depending on the stoichiometric proportions between the charged group(s) in the compound and the counter-ion in the salt, the acid additions salts and/or base addition salts can be either mono-addition salts or poly-addition salts.

The phrase “mono-addition salt”, as used herein, describes a salt in which the stoichiometric ratio between the counter-ion and charged form of the compound is 1:1, such that the addition salt includes one molar equivalent of the counter-ion per one molar equivalent of the compound.

The phrase “poly-addition salt”, as used herein, describes a salt in which the stoichiometric ratio between the counter-ion and the charged form of the compound is greater than 1:1 and is, for example, 2:1, 3:1, 4:1 and so on, such that the addition salt includes two or more molar equivalents of the counter-ion per one molar equivalent of the compound.

An example, without limitation, of a pharmaceutically acceptable salt would be an ammonium cation and an acid addition salt thereof, and/or a hydroxy anion and a base addition salt thereof.

The base addition salts may include a cation counter-ion such as sodium, potassium, ammonium, calcium, magnesium and the like, that forms a pharmaceutically acceptable salt.

The acid addition salts may include a variety of organic and inorganic acids, such as, but not limited to, hydrochloric acid which affords a hydrochloric acid addition salt, hydrobromic acid which affords a hydrobromic acid addition salt, acetic acid which affords an acetic acid addition salt, ascorbic acid which affords an ascorbic acid addition salt, benzenesulfonic acid which affords a besylate addition salt, camphorsulfonic acid which affords a camphorsulfonic acid addition salt, citric acid which affords a citric acid addition salt, maleic acid which affords a maleic acid addition salt, malic acid which affords a malic acid addition salt, methanesulfonic acid which affords a methanesulfonic acid (mesylate) addition salt, naphthalenesulfonic acid which affords a naphthalenesulfonic acid addition salt, oxalic acid which affords an oxalic acid addition salt, phosphoric acid which affords a phosphoric acid addition salt, toluenesulfonic acid which affords a p-toluenesulfonic acid addition salt, succinic acid which affords a succinic acid addition salt, sulfuric acid which affords a sulfuric acid addition salt, tartaric acid which affords a tartaric acid addition salt and trifluoroacetic acid which affords a trifluoroacetic acid addition salt. Each of these acid addition salts can be either a mono-addition salt or a poly-addition salt, as these terms are defined herein.

As used herein, the term “prodrug” describes a compound which is converted in the body to an active compound (e.g., the compound of the formula described hereinabove). A prodrug is typically designed to facilitate administration, e.g., by enhancing absorption. A prodrug may comprise, for example, the active compound modified with ester groups, for example, wherein any one or more of the hydroxyl groups of a compound is modified by an acyl group, optionally (C₁₋₄)-acyl (e.g., acetyl) group to form an ester group, and/or any one or more of the carboxylic acid groups of the compound is modified by an alkoxy or aryloxy group, optionally (C₁₋₄)-alkoxy (e.g., methyl, ethyl) group to form an ester group.

Each of the compounds described herein, including the salts thereof, can be in a form of a solvate or a hydrate thereof.

The term “solvate” describes a complex of variable stoichiometry (e.g., di-, tri-, tetra-, penta-, hexa-, and so on), which is formed by a solute (the heterocyclic compounds described herein) and a solvent, whereby the solvent does not interfere with the biological activity of the solute.

The term “hydrate” describes a solvate, as defined hereinabove, where the solvent is water.

The compounds described herein can be used as polymorphs and the present embodiments further encompass any isomorph of the compounds and any combination thereof.

The compounds and structures described herein encompass any stereoisomer, including enantiomers and diastereomers, of the compounds described herein, unless a particular stereoisomer is specifically indicated.

As used herein, the term “enantiomer” describes a stereoisomer of a compound that is superposable with respect to its counterpart only by a complete inversion/reflection (mirror image) of each other. Enantiomers are said to have “handedness” since they refer to each other like the right and left hand. Enantiomers have identical chemical and physical properties except when present in an environment which by itself has handedness, such as all living systems. In the context of the present embodiments, a compound may exhibit one or more chiral centers, each of which exhibiting an (R) or an (S) configuration and any combination, and compounds according to some embodiments of the present invention, can have any their chiral centers exhibit an (R) or an (S) configuration.

The term “diasterceomers”, as used herein, describes stereoisomers that are not enantiomers to one another. Diastereomerism occurs when two or more stereoisomers of a compound have different configurations at one or more, but not all of the equivalent (related) stereocenters and are not mirror images of each other. When two diastereoisomers differ from each other at only one stereocenter they are epimers. Each stereo-center (chiral center) gives rise to two different configurations and thus to two different stereoisomers. In the context of the present invention, embodiments of the present invention encompass compounds with multiple chiral centers that occur in any combination of stereo-configuration, namely any diastereomer.

The term “tautomer” describes a product of the rapid equilibrium between a carbonyl group (C═O) and its enol tautomer, such that for any enol (e.g., —CH═C(OH)—) a respective keto form (e.g., —CH—C(═O)—) is encompassed, and for any keto, a respective enol form is encompassed. Active derivatives of enterobactin comprise the general formula I and comprise bactericidal and/or bacteriostatic towards at least one gram positive bacteria. The derivative may be a naturally occurring derivative or a synthetic derivative.

Typically, the enterobactin and active derivative thereof is non-toxic to mammals e.g. humans.

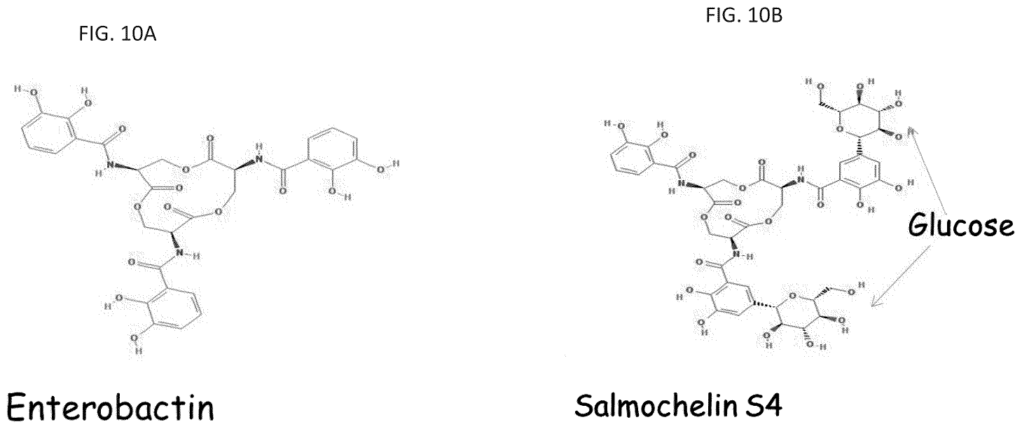

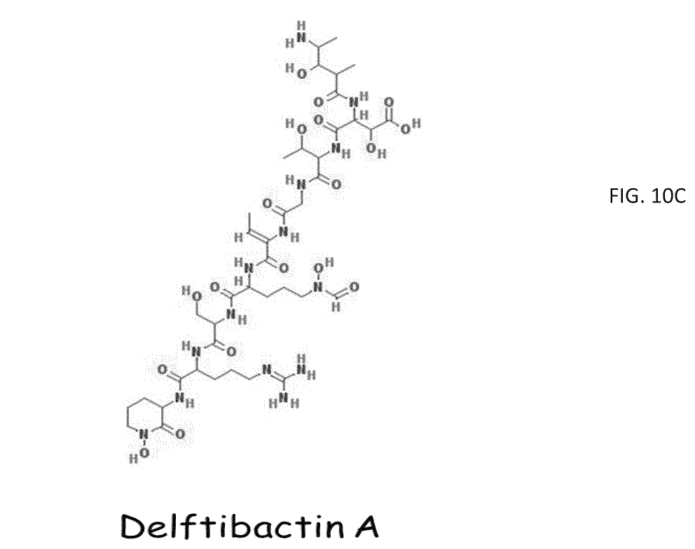

In one embodiment, the enterobactin has the molecular structure as shown in FIG. 10A wherein R₁-R₉ are each hydrogen.

Enterobactin may be derivatized at the 4-, 5-, or 6-position of the one or more catecholate moieties, such that one or more of R₁, R₆ and R₉ is other than hydrogen (position 4 of the catecholate); and/or one or more of R₂, R₅ and R₈ is other than hydrogen (position 5 of the catecholate); and/or one or more of R₃, R₄ and/or R₇ is other than hydrogen (position 6 of the catecholate). For example, enterobactin may be derivatized at the 5-position of the catecholate moiety, which provides a point for site-specific modification without compromising the Fe(III)-binding groups or the macrolactone, such that one or more of R₂, R₅ and R₈ is other than hydrogen.

In some embodiments, one or more of R₁-R₉ is independently a saccharide moiety.

Herein, the phrase “saccharide moiety” encompasses monosaccharides and oligosaccharides, as these terms are defined herein.

The term “monosaccharide”, as used herein and is well known in the art, describes a simple form of a sugar that consists of a single saccharide molecule which cannot be further decomposed by hydrolysis. Most common examples of monosaccharides include glucose (dextrose), fructose, galactose, and ribose. Monosaccharides can be classified according to the number of carbon atoms of the carbohydrate, i.e., triose, having 3 carbon atoms such as glyceraldehyde and dihydroxyacetone; tetrose, having 4 carbon atoms such as erythrose, threose and erythrulose; pentose, having 5 carbon atoms such as arabinose, lyxose, ribose, xylose, ribulose and xylulose; hexose, having 6 carbon atoms such as allose, altrose, galactose, glucose, gulose, idose, mannose, talose, fructose, psicose, sorbose and tagatose; heptose, having 7 carbon atoms such as mannoheptulose, sedoheptulose; octose, having 8 carbon atoms such as 2-keto-3-deoxy-manno-octonate; nonose, having 9 carbon atoms such as sialose; and decose, having 10 carbon atoms. Monosaccharides are the building blocks of oligosaccharides like sucrose (common sugar) and other polysaccharides (such as cellulose and starch).

The term “oligosaccharide” as used herein describes a compound that comprises two or more monosaccharide units, as these are defined herein, linked to one another via a glycosyl bond (—O—). Preferably, the oligosaccharide comprises 2-6 monosaccharides, more preferably the oligosaccharide comprises 2-4 monosaccharides and most preferably the oligosaccharide is a disaccharide moiety, having two monosaccharide units.

When two or more of R₁-R₉ are each a saccharide moiety, the saccharide moiety can be the same or different. In some embodiments, one or more of R₁-R₉ is a monosaccharide.

In some embodiments, one or more of R₁-R₉ is glucose, such that the active derivative is a glucosylated derivative—e.g. comprises one or more glucose moieties attached to the C4, C5 and/or C6 position(s) of one or more catecholate rings of the enterobactin. In one embodiment, the glucosylated derivative comprises at least one glucose moiety attached to C5 position of one or more catecholate rings of the enterobactin, such that one or more of R₂, R₅ and R₈ is glucose. The molecular structure of an exemplary glucosylated derivative (Salmochelin S4) is illustrated in FIG. 10B.

Hydrolyzed enterobactin analogs include linear Ent,2,3-dihydroxybenzyl serine (DHBS) monomer and dimer, and glucosylated forms thereof. See FIGS. 1A-B, which shows the structures of several of these analogs. Non-hydrolyzable enterobactin analogs include: cationic analogs of enterobactin such as tris catecholate analogs lacking macrolactone (see Rodgers et al. Inorg Chem. 1987: 26(10):1622-5; Tor et al. J Am Chem Soc. 1992: 114(17):6653-61; Ecker et al. J. Am Chem Soc. 1988: 110(8): 2457-64; Stack et al. J Am Chem Soc. 1993; 115(14):6466-7: Ji et al. J Am Chem Soc. 2012: 134(24):9898-901).

Enterobactin is commercially available—for example from Sigma-Aldrich (E3910) and from EMC Microcollections (Tübingen, Germany). Salmochelin S4 is commercially available (for example from EMC Microcollections, Tübingen, Germany).

According to a particular embodiment, the enterobactin (or active derivative thereof) is not conjugated to an antibiotic.

According to another embodiment, the enterobactin (or active derivative thereof) is not conjugated to a carrier protein or a linker. In some embodiments, the enterobactin is complexed with iron, i.e., is ferric enterobactin (FeFnt), ferric salmochelin, or an analog thereof. In other embodiments, the enterobactin (or derivative thereof) is not complexed with iron.

The term “Gram-positive bacteria” as used herein refers to bacteria characterized by having as part of their cell wall structure peptidoglycan as well as polysaccharides and/or teichoic acids and are characterized by their blue-violet color reaction in the Gram-staining procedure. Representative Gram-positive bacteria include: Actinomyces spp., Bacillus anthracis, Bifidobacterium spp., Clostridium botulinum, Clostridium perfringens, Clostridium spp., Clostridium tetani, Corynebacterium diphtheriae, Corynebacterium jeikeium, Enterococcus faecalis, Enterococcus faccium, Erysipelothrix rhusiopathiae, Eubacterium spp., Gardnerella vaginalis, Gemella morbillorum, Leuconostoc spp., Mycobacterium abcessus, Mycobacterium avium complex, Mycobacterium chelonae, Mycobacterium fortuitum, Mycobacterium haemophilium, Mycobacterium kansasii, Mycobacterium leprae, Mycobacterium marinum, Mycobacterium scrofulaceum, Mycobacterium smegmatis, Mycobacterium terrae, Mycobacterium tuberculosis, Mycobacterium ulcerans, Nocardia spp., Peptococcus niger, Peptostreptococcus spp., Proprionibacterium spp., Staphylococcus aureus, Staphylococcus auricularis, Staphylococcus capitis, Staphylococcus cohnii, Staphylococcus epidermidis, Staphylococcus haemolyticus, Staphylococcus hominis, Staphylococcus lugdanensis, Staphylococcus saccharolyticus, Staphylococcus saprophyticus, Staphylococcus schleiferi, Staphylococcus similans, Staphylococcus warneri, Staphylococcus xylosus, Streptococcus agalactiae (group B Streptococcus), Streptococcus anginosus, Streptococcus bovis, Streptococcus canis, Streptococcus equi, Streptococcus milleri, Streptococcus mitior, Streptococcus mutans, Streptococcus pneumoniae, Streptococcus pyogenes (group A Streptococcus), Streptococcus salivarius, Streptococcus sanguis.

The present inventors contemplate treating the bacterial infections using enterobactin alone, salmochelin S4 alone or a combination of enterobactin and salmochelin S4. Such combinations were shown to have enhanced anti-bacterial activity (See for example, FIG. 4).

Since combining enterobactin with additional siderophores led to enhanced antimicrobial activity (see for example FIG. 5), the present inventors contemplate administering enterobactin together with other siderophores to treat infection.

In one embodiment, the additional siderophore is a glucosylated derivative of enterobactin—e.g. Salmochelin S4.

In another embodiment, the additional siderophore is a catecholate siderophore including, but not limited to bacillibactin and vibriobactin.

In another embodiment, the additional siderophore is delftibactin A or an analogue thereof, examples of which are provided in U.S. patent application Ser. No. 11/197,907.

Combinations of glucosylated derivative of enterobactin, such as Salmochelin S4 together with other siderophores are also contemplated for the treatment of gram positive bacterial infections.

The enterobactin (or derivative thereof) may be provided together with other antimicrobial agents.

As used herein, the term “antimicrobial agent” refers to an agent having antimicrobial activity—i.e. the ability to suppress, control, inhibit or kill microorganisms, such as bacteria, fungi, viruses, protists and archae.

According to a particular embodiment, the antimicrobial agent is not an antifungal agent.

According to a specific embodiment, the antimicrobial agent is an antibacterial agent—e.g. an antibiotic, an antimicrobial peptide and an antibacterial siderophore.

Examples of antibiotics contemplated by the present invention include, but are not limited to Daptomycin; Gemifloxacin; Telavancin; Ceftaroline; Fidaxomicin; Amoxicillin; Ampicillin; Bacampicillin; Carbenicillin; Cloxacillin; Dicloxacillin; Flucloxacillin; Mezlocillin; Nafcillin; Oxacillin; Penicillin G; Penicillin V; Piperacillin; Pivampicillin; Pivmecillinam; Ticarcillin; Aztreonam; Imipenem; Doripenem; Meropenem; Ertapenem; Clindamycin; Lincomycin; Pristinamycin; Quinupristin; Cefacetrile (cephacetrile); Cefadroxil (cefadroxyl); Cefalexin (cephalexin); Cefaloglycin (cephaloglycin); Cefalonium (cephalonium); Cefaloridine (cephaloradine); Cefalotin (cephalothin); Cefapirin (cephapirin); Cefatrizine; Cefazaflur; Cefazedone; Cefazolin (cephazolin); Cefradine (cephradine); Cefroxadine; Ceftezole; Cefaclor; Cefamandole; Cefmetazole; Cefonicid; Cefotetan; Cefoxitin; Cefprozil (cefproxil); Cefuroxime; Cefuzonam; Cefcapene; Cefdaloxime; Cefdinir; Cefditoren; Cefetamet; Cefixime; Cefmenoxime; Cefodizime; Cefotaxime; Cefpimizole; Cefpodoxime; Cefteram; Ceftibuten; Ceftiofur; Ceftiolene; Ceftizoxime; Ceftriaxone; Cefoperazone; Ceftazidime; Cefclidine; Cefepime; Cefluprenam; Cefoselis; Cefozopran; Cefpirome; Cefquinome; Fifth Generation; Ceftobiprole; Ceftaroline; Not Classified; Cefaclomezine; Cefaloram; Cefaparole; Cefcanel; Cefedrolor; Cefempidone; Cefetrizole; Cefivitril; Cefmatilen; Cefmepidium; Cefovecin; Cefoxazole; Cefrotil; Cefsumide; Cefuracetime; Ceftioxide; Azithromycin; Erythromycin; Clarithromycin; Dirithromycin; Roxithromycin; Telithromycin; Amikacin; Gentamicin; Kanamycin; Neomycin; Netilmicin; Paromomycin; Streptomycin; Tobramycin; Flumequine; Nalidixic acid; Oxolinic acid; Piromidic acid; Pipemidic acid; Rosoxacin; Ciprofloxacin; Enoxacin; Lomefloxacin; Nadifloxacin; Norfloxacin; Ofloxacin; Pefloxacin; Rufloxacin; Balofloxacin; Gatifloxacin; Grepafloxacin; Levofloxacin; Moxifloxacin; Pazufloxacin; Sparfloxacin; Temafloxacin; Tosufloxacin; Besifloxacin; Clinafloxacin; Gemifloxacin; Sitafloxacin; Trovafloxacin; Prulifloxacin; Sulfamethizole; Sulfamethoxazole; Sulfisoxazole; Trimethoprim-Sulfamethoxazole; Demeclocycline; Doxycycline; Minocycline; Oxytetracycline; Tetracycline; Tigecycline; Chloramphenicol; Metronidazole; Tinidazole; Nitrofurantoin; Vancomycin; Teicoplanin; Telavancin; Linezolid; Cycloserine 2; Rifampin; Rifabutin; Rifapentine; Bacitracin; Polymyxin B; Viomycin; Capreomycin.

According to a particular embodiment, the antibiotic is lincomycin.

In one preferred embodiment, the amount of the anti-microbial agent (when used in combination with the enterobactin, or active derivative thereof) is below the minimum dose required for therapeutic or prophylactic effectiveness when used as a single therapy (e.g. 10-99%, preferably 25 to 75% of that minimum dose). This allows for reduction of the side effects caused by the anti-microbial agent but the therapy is rendered effective because in combination with the enterobactin or derivative thereof of the present invention, the combinations are effective overall. Alternatively, the combination of the two agents may allow for long-term use without building up resistance.

In another embodiment, the amount of the enterobactin, or active derivative thereof (when used in combination with the anti-microbial agent) is below the minimum dose required for therapeutic or prophylactic effectiveness when used as a single therapy (e.g. 10-99%, preferably 25 to 75% of that minimum dose). This allows for reduction of the side effects caused by the enterobactin, or active derivative thereof but the therapy is rendered effective because in combination with the anti-microbial agent, the combinations are effective overall.

According to one embodiment, the anti-microbial agent is administered prior to the enterobactin (or active derivative thereof). In another embodiment, the anti-microbial agent is administered following administration of the enterobactin (or active derivative thereof).

In still another embodiment, the anti-microbial agent is administered concomitantly with the enterobactin or active derivative thereof. In this embodiment, the anti-microbial agent may be administered in the same composition (i.e. co-formulated in a single composition, or may be coated on the same surface, as further described herein below) as the enterobactin, or active derivative thereof.

Since it is known that siderphores bind with high affinity to metals such as gold or gallium, the present inventors further contemplate using enterobactin (or derivative thereof) which is attached to such metals for enhancing its antimicrobial properties. Both gallium and gold are known for their antimicrobial properties—see for example Antunes et al., Antimicrob. Agents Chemother. November 2012 vol. 56 no. 11 5961-5970; and Zhou et al., J Nanobiotechnology. 2012; 10: 19.

Particular combinations of agents which can be used as effective antimicrobial agents include the following:
(i) enterobactin and a glucosylated derivative thereof (e.g. Salmochelin S4); and
(ii) enterobactin and delftibactin;
(iii) Salmochelin S4 and delftibactin;
(iv) enterobactin and a metal;
(v) Salmochelin S4 and a metal
(vi) enterobactin and an antibiotic;
(vii) Salmochelin S4 and an antibiotic.

As well as targeting gram positive bacteria, the combinatory treatment described herein above, may also be used as antibacterial agents against gram negative bacteria.

The term “Gram-negative bacteria” as used herein refer to bacteria characterized by the presence of a double membrane surrounding each bacterial cell. Representative Gram-negative bacteria include Acinetobacter calcoaceticus, Actinobacillus actinomycetemcomitans, Aeromonas hydrophila, Alcaligenes xylosoxidans, Bacteroides, Bacteroides fragilis, Bartonella bacilliformis, Bordetella spp., Borrelia burgdorferi, Branhamella catarrhalis, Brucella spp., Campylobacter spp., Chalmydia pneumoniae, Chlamydia psittaci, Chlamydia trachomatis, Chromobacterium violaceum, Citrobacter spp., Eikenella corrodens, Enterobacter aerogenes, Escherichia coli, Flavobacterium meningosepticum, Fusobacterium spp., Haemophilus influenzae, Haemophilus spp., Helicobacter pylori, Klebsiella spp., Legionella spp., Leptospira spp., Moraxella catarrhalis, Morganella morganii, Mycoplasma pneumoniae, Neisseria gonorrhoeae, Neisseria meningitidis, Pasteurella multocida, Plesiomonas shigelloides, Prevotella spp., Proteus spp., Providencia rettgeri, Pseudomonas aeruginosa, Pseudomonas spp., Rickettsia prowazekii, Rickettsia rickettsii, Rochalimaea spp., Salmonella spp., Salmonella typhi, Serratia marcescens, Shigella spp., Treponema carateum, Treponema pallidum, Treponema pallidum endemicum, Treponema pertenue, Veillonella spp., Vibrio cholerae, Vibrio vulnificus, Yersinia enterocolitica, Yersinia pestis.

The enterobactin and/or derivative thereof (and optional additional anti-microbial agent) may be administered per se, or as part of a pharmaceutical composition.

The phrase “pharmaceutical composition”, as used herein refers to a preparation of one or more of the active ingredients described herein with other chemical components such as physiologically suitable carriers and excipients. The purpose of a pharmaceutical composition is to facilitate administration of a compound to an organism.

As used herein the term “active ingredient” refers to the agents of the present invention accountable for the intended biological effect.

Hereinafter, the phrases “physiologically acceptable carrier” and “pharmaceutically acceptable carrier”, which may be used interchangeably, refer to a carrier or a diluent that does not cause significant irritation to an organism and does not abrogate the biological activity and properties of the administered compound. An adjuvant is included under these phrases.

Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils, and polyethylene glycols.

Techniques for formulation and administration of drugs may be found in the latest edition of “Remington's Pharmaceutical Sciences”, Mack Publishing Co., Easton, PA, which is herein fully incorporated by reference and are further described herein below.

It will be appreciated that the agents of the present invention can be provided to the individual with additional active agents to achieve an improved therapeutic effect as compared to treatment with each agent by itself.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the invention may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The preparations described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The preparation of the present invention may also be formulated in rectal compositions such as suppositories or retention enemas, using, e.g., conventional suppository bases such as cocoa butter or other glycerides.

The preparation of the present invention may also be formulated as a topical compositions, such as a spray, a cream, a mouthwash, a wipe, a foam, a soap, an oil, a solution, a lotion, an ointment, a paste and a gel.

Pharmaceutical compositions suitable for use in context of the present invention include compositions wherein the active ingredients are contained in an amount effective to achieve the intended purpose. More specifically, a therapeutically effective amount means an amount of active ingredients effective to prevent, alleviate or ameliorate symptoms of disease or prolong the survival of the subject being treated.

Determination of a therapeutically effective amount is well within the capability of those skilled in the art.

For any preparation used in the methods of the invention, the therapeutically effective amount or dose can be estimated initially from in vitro assays. For example, a dose can be formulated in animal models and such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. [See e.g., Fingl, et al., (1975) “The Pharmacological Basis of Therapeutics”, Ch. 1 p. 1].

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

As well as for treating infections, the present inventors contemplate using the enterobactin and/or active derivative for killing bacteria on surfaces. Bacteria present on surfaces are contacted with the above described compositions under conditions that bring a down-regulation in an amount and/or activity of the bacteria.

As used herein the term “contacting” refers to the positioning of the agents of the present invention such that they are in direct or indirect contact with the bacterial cells. Thus, the present invention contemplates both applying the agents of the present invention to a desirable surface and/or directly to the bacterial cells.

Contacting surfaces with the agents described herein can be effected using any method known in the art including spraying, spreading, wetting, immersing, dipping, painting, ultrasonic welding, welding, bonding or adhering. The agents of the present invention may be attached to a solid surface as monolayers or multiple layers.

The present invention envisages coating a wide variety of surfaces with the compositions of the present invention including fabrics, fibers, foams, films, concretes, masonries, glass, metals, plastics, polymers, and like.

An exemplary solid surface that may be coated with the compositions of the present invention is an intracorporial or extra-corporeal medical device or implant.

An “implant” as used herein refers to any object intended for placement in a human body that is not a living tissue. The implant may be temporary or permanent. Implants include naturally derived objects that have been processed so that their living tissues have been devitalized. As an example, bone grafts can be processed so that their living cells are removed (acellularized), but so that their shape is retained to serve as a template for ingrowth of bone from a host. As another example, naturally occurring coral can be processed to yield hydroxyapatite preparations that can be applied to the body for certain orthopedic and dental therapies. An implant can also be an article comprising artificial components.

Thus, for example, the present invention therefore envisions coating vascular stents with the agents of the present invention. Another possible application of the agents of the present invention is the coating of surfaces found in the medical and dental environment.

Surfaces found in medical environments include the inner and outer aspects of various instruments and devices, whether disposable or intended for repeated uses. Examples include the entire spectrum of articles adapted for medical use, including scalpels, needles, scissors and other devices used in invasive surgical, therapeutic or diagnostic procedures; blood filters, implantable medical devices, including artificial blood vessels, catheters and other devices for the removal or delivery of fluids to patients, artificial hearts, artificial kidneys, orthopedic pins, plates and implants; catheters and other tubes (including urological and biliary tubes, endotracheal tubes, peripherally insertable central venous catheters, dialysis catheters, long term tunneled central venous catheters peripheral venous catheters, short term central venous catheters, arterial catheters, pulmonary catheters, Swan-Ganz catheters, urinary catheters, peritoneal catheters), urinary devices (including long term urinary devices, tissue bonding urinary devices, artificial urinary sphincters, urinary dilators), shunts (including ventricular or arterio-venous shunts); prostheses (including breast implants, penile prostheses, vascular grafting prostheses, aneurysm repair devices, heart valves, artificial joints, artificial larynxes, otological implants), anastomotic devices, vascular catheter ports, clamps, embolic devices, wound drain tubes, hydrocephalus shunts, pacemakers and implantable defibrillators, and the like. Other examples will be readily apparent to practitioners in these arts.

Surfaces found in the medical environment include also the inner and outer aspects of pieces of medical equipment, medical gear worn or carried by personnel in the health care setting. Such surfaces can include counter tops and fixtures in areas used for medical procedures or for preparing medical apparatus, tubes and canisters used in respiratory treatments, including the administration of oxygen, of solubilized drugs in nebulizers and of anesthetic agents. Also included are those surfaces intended as biological barriers to infectious organisms in medical settings, such as gloves, aprons and faceshields. Commonly used materials for biological barriers may be latex-based or non-latex based. Vinyl is commonly used as a material for non-latex surgical gloves. Other such surfaces can include handles and cables for medical or dental equipment not intended to be sterile. Additionally, such surfaces can include those non-sterile external surfaces of tubes and other apparatus found in areas where blood or body fluids or other hazardous biomaterials are commonly encountered.

Other surfaces related to health include the inner and outer aspects of those articles involved in water purification, water storage and water delivery, and those articles involved in food processing. Thus the present invention envisions coating a solid surface of a food or beverage container to extend the shelf life of its contents.

Surfaces related to health can also include the inner and outer aspects of those household articles involved in providing for nutrition, sanitation or disease prevention. Examples can include food processing equipment for home use, materials for infant care, tampons and toilet bowls.

In addition, the agents of the present invention may have veterinary applications including disinfection of animal cages, coops or homes.

As used herein the term “about” refers to +10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Example 1

Antibacterial Effect of Enterobactin and Salmochelin against Gram Positive Bacteria

### Materials and Methods

Iron-free enterobactin from E. coli was purchased from Sigma-Aldrich (E3910) and from EMC (Tübingen, Germany), iron-free salmochelin S4 was purchased from EMC (Tübingen, Germany).

Bacterial strains were provided by the Clinical Microbiology Laboratory at Sheba Medical Center.

Inhibitory effect was measured using broth microdilution according to Clinical and Laboratory Standards Institute (CLSI) guidelines, in 96-well microtiter plate, with cation-adjusted Mueller-Hinton Broth (CAMHB), and using Tecan plate reader. Bacterial final inoculum was 5×10⁵ colony-forming units ml⁻¹. Enterobactin and salmochelin were dissolved in 100% dimethylsulfoxide (DMSO) to a stock solution of 10 μM and further diluted using CAMHB to final concentrations.

### Results

As illustrated in FIGS. 1A-B both Salmochelin and Enterobactin have an inhibitory effect on clinical isolates of MRSA USA300.

As summarized below, Salmochelin (Table 1A) and Enterobactin (Table 1B) has inhibitory effects on other clinical S. aureus isolates.

TABLE 1A
Summary of the inhibitory effect of salmochein
on different clinical S. aureus isolates.

| | Salmochelin concentration (μM) | |
|---|---|---|
| Bacteria | which inhibits 50% (or more) growth | |
| S. aureus ATCC 25923 | 2.4 ± 0.22 | n = 5 |
| MRSA SA 742 USA300 | 3.125 ± 1.25 | n = 5 |
| MRSA SA 196 | 2.5 | |
| MSSA SA 197 USA300 | 2.5 | |
| MRSA SA 198 USA300 | 5 | |
| MRSA SA 255 | 5 | |
| MSSA SA 256 | 5 | |

TABLE 1B
Summary of the inhibitory effect of enterobactin
on different clinical S. aureus isolates.

| | Enterobactin concentration (μM) | |
|---|---|---|
| Bacteria | which inhibits 50% (or more) growth | |
| S. aureus ATCC 25923 | 6.0 ± 2.7 | n = 4 |
| MRSA USA300 SA 742 | 10 | n = 4 |
| MRSA-SA 196 | 5 | |
| MSSA SA 197 USA300 | >5 | |
| MRSA SA 198 | >5 | |
| MRSA SA 255 | >5 | |
| MSSA SA 256 | >5 | |

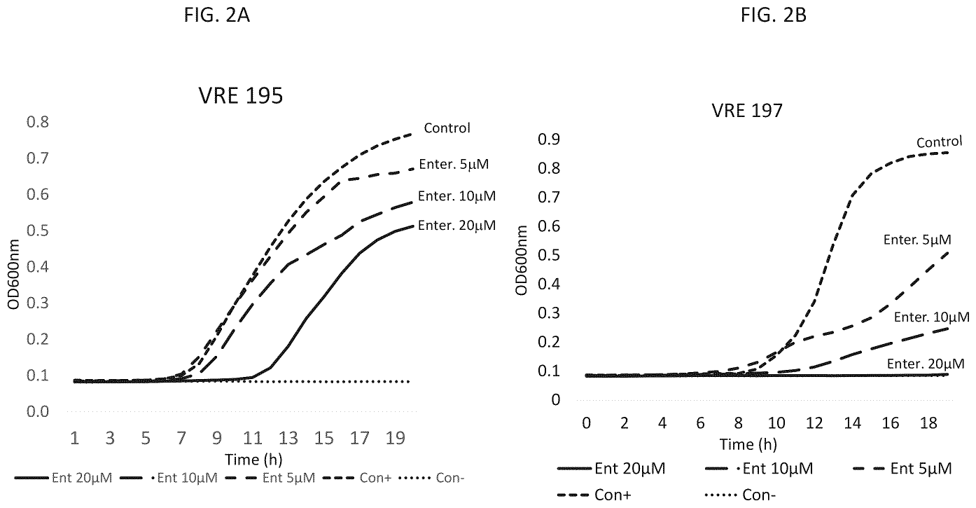

Enterobactin has an inhibitory effect on clinical isolates of VRE 195 (FIG. 2A) and VRE 197 (FIG. 2B).

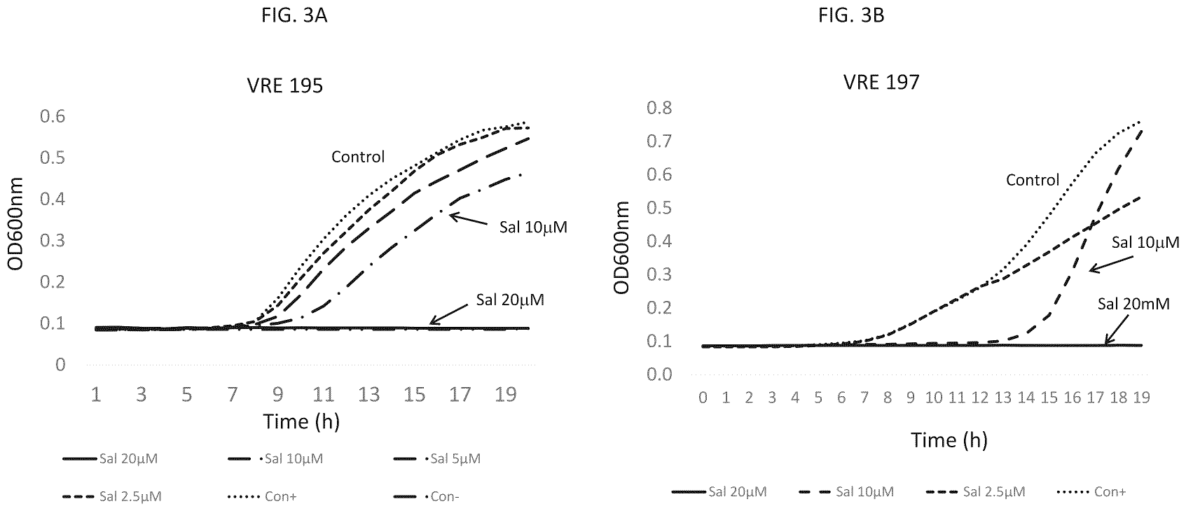

Salmochelin has an inhibitory effect on clinical isolates of VRE 195 (FIG. 3A) and VRE 197 (FIG. 3B).

The combination of Salmochelin and Enterobactin displays enhanced activity against S. aureus—see FIG. 4.

The combination of Delftibactin A and Enterobactin displays enhanced activity against S. aureus—FIG. 5.

As illustrated in FIGS. 6A-B, iron ions reduce but do not abolish antimicrobial activity of salmochelin and enterobactin against S. aureus.

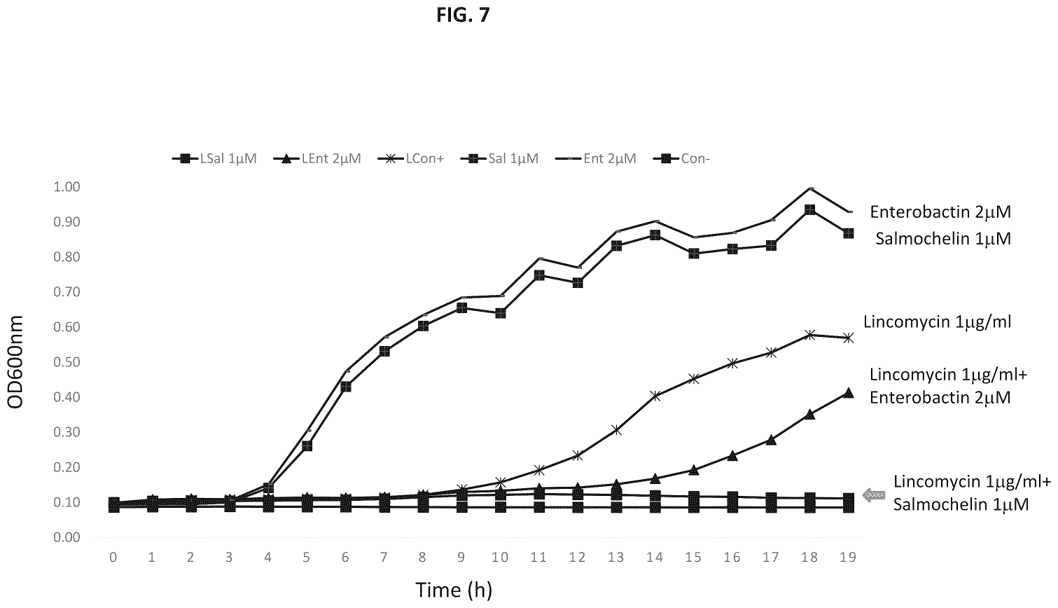

The combination of Salmochelin or Enterobactin with lincomycin displays enhanced activity against S. aureus—FIG. 7.

FIG. 8 illustrates the effect of gold plus Enterobactin or Salmochelin on E. coli CFT073.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A method of treating an infection of a gram positive bacteria in a subject in need thereof, the method comprising administering to the subject a therapeutically effective amount of an enterobactin represented by Formula I: 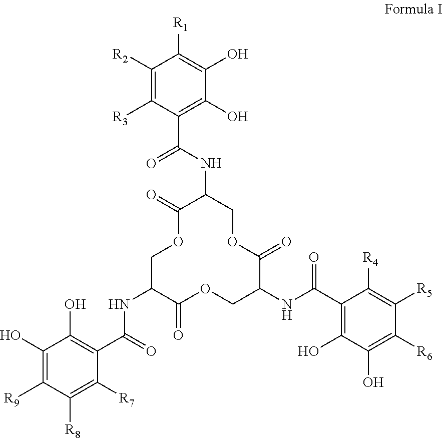
wherein:
R₁-R₉ are each independently selected from hydrogen, alkyl, cycloalkyl, aryl, heteroaryl, heteroalicyclic, halo, alkoxy, aryloxy, thiohydroxy, thioalkoxy, thioaryloxy, and a saccharide moiety, wherein at least 2 of R₂, R₅ and R₈ are each independently said saccharide moiety
and wherein said enterobactin is not conjugated to an antibiotic, thereby treating the infection.

2-3. (canceled)

4. The method of claim 1, wherein the gram positive bacteria is of the genus Staphylococcus or Enterococcus.

5. The method of claim 1, further comprising administering to the subject an antimicrobial agent distinct of said enterobactin.

6. The method of claim 5, wherein said antimicrobial agent is selected from the group consisting of an antibiotic, a metal and delftibactin.

7-23. (canceled)

24. A method of killing a bacteria, the method comprising contacting the bacteria with a composition comprising:
(i) an enterobactin represented by Formula I: 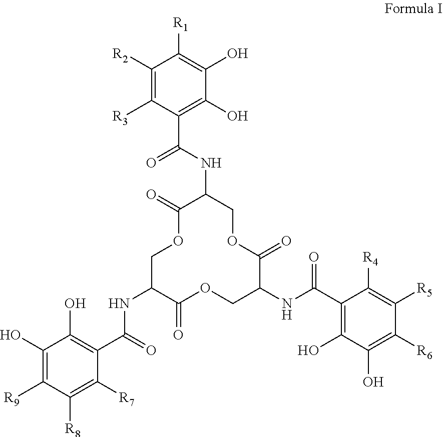
wherein:
R₁-R₉ are each independently selected from hydrogen, alkyl, cycloalkyl, aryl, heteroaryl, heteroalicyclic, halo, alkoxy, aryloxy, thiohydroxy, thioalkoxy, thioaryloxy, and a saccharide moiety, wherein at least 2 of R₂, R₅ and R₈ are each independently said saccharide moiety
and wherein said enterobactin is not conjugated to an antibiotic; and
(ii) a metal or an antibiotic,
thereby killing the bacteria.

25. The method of claim 24, wherein said contacting is effected in vivo.

26. The method of claim 24, wherein said contacting is effected ex vivo.

27. The method of claim 24, wherein the bacteria is a gram positive bacteria.

28-30. (canceled)

31. The method of claim 1, wherein R₁-R₄, R₆, R₇ and R₉ are each hydrogen.

32. The method of claim 1, wherein R₅ and R₈ are each independently said saccharide moiety.

33. The method of claim 1, wherein each of said saccharide moieties is glucose.

34. The method of claim 1, wherein:
R₁-R₄, R₆, R₇ and R₉ are each hydrogen; and
R₅ and R₈ are each glucose,
the compound being Salmochelin S4.

35. The method of claim 24, wherein R₁-R₄, R₆, R₇ and R₉ are each hydrogen.

36. The method of claim 24, wherein R₅ and R₈ are each independently said saccharide moiety.

37. The method of claim 24, wherein each of said saccharide moieties is glucose.

38. The method of claim 24, wherein:
R₁-R₄, R₆, R₇ and Ry are each hydrogen; and
R₅ and R₈ are each glucose,
the compound being Salmochelin S4.
